(19) United States
(12) Patent Application Publication
(10) Pub. No.: US 2009/0182064 A1
(43) Pub. Date: Jul. 16, 2009

(54) Reactive Separation To Upgrade Bioprocess Intermediates To Higher Value Liquid Fuels or Chemicals

(75) Inventor: Thomas Paul Griffin, Chadds Ford, PA (US)

Correspondence Address: GOODWIN PROCTER LLP, 901 NEW YORK AVENUE, N.W., WASHINGTON, DC, 20001, US

(73) Assignee: Pennsylvania Sustainable Technologies, LLC

(21) Appl. No.: 12/353,654

(22) Filed: Jan. 14, 2009

Related U.S. Application Data
(60) Provisional application No. 61/020,867, filed on Jan. 14, 2008.

(51) Int. Cl. C07C 27/26; C07C 35/00

(52) U.S. Cl. 518/711; 568/700

(57) ABSTRACT

The process and system of the embodiments utilize a reactive separation unit to upgrade a bioprocess intermediate stream to higher value liquid fuels or chemicals. The reactive separation unit simultaneously enables molecular weight and density increases, oxygen content reduction, efficient process energy integration, optional water separation for potential reuse, and incorporation of additional hydrocarbons or oxygenated hydrocarbons as co-feed(s). The use and selection of particular co-feed(s) for this purpose enables tailoring of the intended product composition. The process and system yields a product of higher alcohols, liquid hydrocarbons, or a combination of these. These can be split into two (or more) boiling point fractions by the same reactive separations unit operation resulting in product(s) that can be used as chemicals, chemical intermediates, or alternative (non-fossil-based) liquid transportation fuels.

This application claims priority to provisional application Ser. No. 61/020867, entitled: “Reactive Separation as a Means of Upgrading Bioprocess Intermediates to Higher Value Liquid Fuels or Chemicals,” filed on Jan. 14, 2008, the disclosure of which is incorporated by reference. This application also is related to co-pending patent application Ser. No. ______ , entitled: “Method and System for Producing Alternative Liquid Fuels of Chemicals,” Docket No. PST-002, filed concurrently herewith, the disclosure of which is incorporated herein by reference.

### BACKGROUND

1. Field of the Invention

The embodiments relate to processes and systems for upgrading bioprocess intermediates into higher value liquid fuels or chemicals. One example of an application for the embodiments is to upgrade diluted bioethanol into higher alcohol(s) (C₂+) and/or aliphatic liquid hydrocarbon(s) (C₄+) for use as fuel components or fuel substitutes.

2. Description of the Related Art

Alternative, non-petroleum-based, liquid transportation fuels could provide economic, security, and environmental benefits. Increased worldwide energy demands are likely to increase oil and fuel prices and may motivate new political conflicts. Carbon-based greenhouse gas emissions continue to accumulate in the atmosphere, and the industrialization of populous countries, such as China and India, likely will accelerate that accumulation. Transportation fuels derived from locally available, low-cost inputs could reduce or slow the growth in demand for crude oil, and help to mitigate these problems.

Transportation fuels derived from renewable biomass, or “Biofuels,” are of particular commercial interest. Biomass can be viewed as intermediate-term storage of solar energy and atmospheric carbon, via photosynthesis and carbon fixing mechanisms. With cultivation and harvesting cycles measured in months, biomass is, in principle, a renewable domestic energy resource.

Bioethanol is a popular biofuel. However, bioethanol's chemical and physical property deficiencies relative to conventional combustion fuels such as gasoline limit its attractiveness as a fuel. The volumetric energy density of ethanol is approximately 70% of typical unleaded gasoline products. In addition, the volatility and fugitive loss potential of ethanol is considerably higher. Further, most automobiles have not been modified to run on bioethanol as a stand-alone fuel. Thus, bioethanol's use is currently limited to a low-percentage gasoline additive.

There are significant drawbacks involved with bioethanol production as well. Bioethanol production is challenged by its low “energy payback ratio,” that has historically been close to, or below one, which is the energy break-even point. Pimentel, D., “Ethanol Fuels: Energy Balance, Economics, and Environmental Impacts are Negative”, Natural Resources Research, 12, vol. 2, 127-134 (2003). Specifically, the amount of energy yielded by bioethanol does not significantly exceed the amount of energy consumed in producing it (i.e., cultivating, harvesting, transporting, processing and handling). The overall energy payback ratio of bioethanol production can be improved by reducing the amount of energy required for production.

Moreover, bioprocess operations, such as carbohydrate fermentation to yield bioethanol, often involve the handling and processing of a great deal of water. For example, the amount of water in the fermentation broth of existing bioethanol processes is typically 85% or greater. Process water needs to be thermally sterilized prior to recycle and treated prior to any discharge from the process. Efficient process water management therefore is important.

Gracey and Bolton have disclosed the use of reactive distillation, a method of reactive separation, in the synthesis of light olefins from alcohols, referenced here for its intent of energy integration and process simplification. Gracey, B. P. and L. W. Bolton, “Reactive Distillation for the Dehydration of Mixed Alcohols”, International Application under the Patent Cooperation Treaty (PCT), WO 2007/003899 A1; PCT Publ. Date Jan. 11, 2007, the disclosure of which is incorporated herein in its entirety.

A number of reaction pathways are available for liquids upgrading that use syngas as a reactant; most can be summarized in broad mechanistic groupings. Reformation of syngas alone to aliphatic liquid hydrocarbons suitable for various fuel applications, for example, was first pioneered by Fischer and Tropsch (“F-T”) nearly a century ago. This chemistry has been commercially practiced for decades, most notably by SASOL (South Africa). The importance and potential of the Fischer-Tropsch and related syntheses for fuel derivation from biomass, including current industrial efforts to pursue these routes commercially, are detailed in the comprehensive review of Spath and Dayton of the National Renewable Energy Laboratory (NREL). Spath, P. L. and D. C. Dayton, Preliminary Screening—Technical and Economic Assessment of Synthesis Gas to Fuels and Chemicals with Emphasis on the Potential for Biomass-Derived Syngas; NREL/TP-510-34929, December 2003.

A separate but related category of syngas reactions that has liquid fuel or chemical generation utility is higher alcohols synthesis. The expression “higher alcohols” typically refers to alcohols heavier than methanol, or C₂+alcohols. In addition, these higher alcohols can be accessed by catalytic mechanisms that are similar to (and derived from) the Fischer-Tropsch route.

One such higher alcohol pathway that has been investigated is the “aldol coupling with oxygen retention reversal” mechanism, documented by Nunan et al., among others. Nunan, J. G., R. G. Herman and K. Klier, “Higher Alcohol and Oxygenate Synthesis over Cs/Cu/ZnO/M₂O₃ (M=Al, Cr) Catalysts”, Journal of Catalysis, 116; 222-229 (1989). In this route, higher alcohols are generated from syngas via sequential chain growth of smaller, primary alcohols, which undergo condensation with dehydration. The analogous condensation reaction between methanol and ethanol also is of interest because of established routes to each reactant from biomass, and is described as the Guerbet Reaction pathway, yielding propanol and heavier alcohols via the “Higher Alcohol Biorefinery” concept of Olson et al. Olson, E. S., R. K. Sharma and T. R. Aulich, “Higher Alcohols Biorefinery—Improvement of Catalyst for Ethanol Conversion,” Applied Biochemistry and Biotechnology, 115; 913-932 (2004).

Miller et al. describe the synthesis of higher alcohols from syngas over a mixed Cu—Cr oxide catalyst (without integrated product separation). Miller, J. T. et al., “Catalytic process for producing olefins or higher alcohols from synthesis gas”, U.S. Pat. No. 5,169,869; Apr. 28, 1992, the disclosure of which is incorporated herein in its entirety. Earlier, Quarderer et al. described the use of “lower alcohols” and syngas to generate higher alcohols, specifically over a Mo-based catalyst—without specifying equipment or reaction engineering details. Quarderer, D. J. et al., “Preparation of ethanol and higher alcohols from lower carbon number alcohols”, U.S. Pat. No. 4,825,013; Apr. 25, 1989, the disclosure of which is incorporated herein in its entirety. Similarly, Landis et al. described the pursuit of two product types in tandem, from FT routes—broadly in terms of hydrocarbons and oxygenates. Landis, S. R. et al., “Managing hydrogen and carbon monoxide in a gas to liquid plant to control the H₂/CO ratio in the Fischer-Tropsch reactor feed”, U.S. Pat. No. 6,872,753; Mar. 29, 2005, the disclosure of which is incorporated herein in its entirety.

Several Conoco patents describe reactive separation as associated with F-T syntheses. For example, Espinoza et al. describes the construct of an F-T catalyst structure on oxide supports (e.g., alumina) with reactive distillation as a possible operation associated with this catalyst. Espinoza, R. L., “Supports for high surface area catalysts”, U.S. Pat. No. 7,276,540; Oct. 2, 2007, the disclosure of which is incorporated herein in its entirety. Two patents by Zhang et al. describe water removal associated with similar catalytic F-T operations, also with mention of reactive distillation as a processing option. Zhang, J. et al., “Method for reducing water concentration in a multi-phase column reactor”, U.S. Pat. No. 6,956,063; Oct. 18, 2005; and Zhang, J. et al., “Water removal in Fischer-Tropsch processes”, U.S. Pat. No. 7,001,927; Feb. 21, 2006, the disclosures of which are incorporated herein in their entirety. Chao et al. discloses similar operations, further specifying the capability to generate C₅+hydrocarbons via this F-T operation with optional reactive distillation. Chao, W. et al., “Fischer-Tropsch processes and catalysts with promoters”, U.S. Pat. No. 6,759,439; Jul. 6, 2004, the disclosure of which is incorporated herein in its entirety.

Some believe that a heavier range of fuel components, including both hydrocarbons and simple (mono) alcohols could offer superior fuel performance to bioethanol. Significant biofuels research and development efforts therefore are being devoted to this hypothesis. For example, DuPont and BP have announced the pursuit of biological routes to butanol (“biobutanol”) as a preferred fuel supplement. The superior fuel performance of butanol relative to ethanol has been quantitatively supported by fuel property testing results. BP Corporation Press Release, “Test Results Show Biobutanol Performs Similarly to Unleaded Gasoline”, BP Corporation Press Release, Apr. 20, 2007; archived via Green Car Congress website: http://www.greencarcongress.com/2007/04/test results sh.html#more. Even heavier alcohols (i.e., heavier than butanol)—and analogous hydrocarbons—are expected to be even better fuel replacements. For example, mixtures of aliphatic hydrocarbons and some higher alcohol and/or ether species would be a more desirable alternative fuel mixture for today's automotive engines. The advantages of such fuel mixtures also have been disclosed by Jimeson et al. (Standard Alcohol Company of America). Jimeson, R. M., Radosevich, M. C., and Stevens, R. R., “Mixed Alcohol Fuels for Internal Combustion Engines, Furnaces, Boilers, Kilns and Gasifiers”, International Application under the Patent Cooperation Treaty (PCT), WO 2006/088462 A1; PCT Publ. Date Aug. 24, 2006, the disclosure of which is incorporated herein in its entirety.

### SUMMARY OF EXEMPLARY EMBODIMENTS

One embodiment uses a reactive separation unit operation to upgrade a bioprocess product intermediate to a more valuable liquid fuel or chemical feedstock. A feature of the invention is the utilization of a second feed stream in the separation process. This second stream is an additional chemical or fuel intermediate in the form of carbon monoxide, hydrogen, syngas, or alcohol(s), or other oxygenated hydrocarbon(s), or any combination of these. This allows the integration of the liquids upgrading reactions with product separations; accomplished directly by the reactive separation operation. In biofuels upgrading for example, this mitigates two resource utility shortcomings; it improves energy payback and facilitates the efficient removal of process water for reuse.

In yet another exemplary aspect of the invention, reactive distillation is utilized as the separating process to upgrade the chemical or fuel value of a bioprocessing intermediate along with a separately-sourced syngas, CO, H₂, or other bioprocessing intermediate (or any combination thereof). With this second feed, reactive distillation affords intraprocess energy and water management integration.

In yet another exemplary aspect of the invention, the mechanism for higher alcohol generation is catalytic alcohol condensation with water rejection, or a catalytic aldol coupling mechanism, also with water rejection. If higher hydrocarbon is the desired product, the mechanism is a catalytic Fischer-Tropsch mechanism. Both the desired molecular weight growth and oxygen removal are initiated via dehydration reactions in a heterogeneous catalytic reaction zone or stage.

The hydrocarbons or oxygenated hydrocarbons are initially concentrated through water removal. The resultant hydrocarbon-rich phase continues to react in the rectification zone(s) of the integrated reactive separation, either through the same reactions or additional chain-growth, dehydration synthesis reactions. The exotherm generated by the higher alcohol synthesis and/or the Fischer-Tropsch synthesis reaction(s), along with a portion of the energy from upstream gasification—carried with the syngas intermediate—drives the reactive separation operations and provides the energy required for the continuous separation.

In yet another exemplary aspect of the invention, the process utilizes parallel reactive separation schemes to produce either an oxygenated liquid (e.g., higher alcohols, C₂₊ primary, secondary, or tertiary saturated alcohols or any combination of these), higher density aliphatic liquid hydrocarbons (C₄₊ saturated, straight-chain or branched aliphatic hydrocarbons or any combination of these), or a combination of these classes depending upon the reactive separation scheme chosen. If desired, the products can be recombined in appropriate ratio(s) to achieve a specified chemical or fuel mixture composition.

In yet another exemplary aspect of the invention, the embodiments also allow for two or more boiling point fractions of each product type to be drawn (via side streams) from the rectification stage(s).

In yet another exemplary aspect of the invention, the separation process can utilize one or more of the following to remove the water-rich phase in order to control the desired output: a slurry or other mixed heterogeneous catalytic reaction zone, a hydrothermal pressure stage for initial handling of stream(s) that still contain a significant amount of water, provision for controlled pressure drop or isenthalpic flash in tandem with the water removal and product rectification stages, a reactive separations stage that accomplishes removal of a water-rich phase, and a rectification section of the reactive separations operation—including one or more equilibrium stage(s).

It is to be understood that both the foregoing general description of the embodiments and the following detailed description are exemplary, but are not restrictive, of the invention. It also is understood that the description in this section of various features, disadvantages, or advantages of known systems, methods, etc., does not mean that one or more of these known systems, methods, etc., are or are not utilized in the embodiments. Indeed, certain features of the embodiments may include known methods or systems without suffering from the disadvantages mentioned herein.

### BRIEF DESCRIPTION OF THE DRAWINGS

A more complete appreciation of the invention and many attendant advantages thereof will be readily obtained as the same becomes better understood by reference to the following detailed description when considered in connection with the accompanying drawing, in which:

FIG. 1 illustrates a schematic representation of the reactive separation unit for upgrading bioprocess intermediates to higher value liquid fuels or chemicals.

### DETAILED DESCRIPTION

Throughout this description, the expression “bioprocess output stream” denotes a stream (fluid, solid, or gas) from a bioprocess unit operation including, but not limited to, fermentation unit operations, aerobic or anaerobic digestion processes, processes using biological materials (e.g., bugs, bacteria, viruses, etc.) to convert organic or other cellulosic-containing materials into useful materials; solvent, acid, or base treatment of cellulosic- or lignocellulosic-containing materials, or other chemical or biochemical treatment or pretreatment of biomass or biomass-containing materials, mixtures, or solutions. The “bioprocess output stream” preferably includes at least a “hydrocarbon product” or an “oxygenated hydrocarbon product.”

Throughout this description, the expressions “hydrocarbon product” or “oxygenated hydrocarbon product” denote products of a bioprocess that have at least one hydrogen atom and one carbon atom, or products of a bioprocess that have at least one hydrogen atom and one carbon atom in which at least one hydrogen atom has been replaced with an oxygen-containing moiety, respectively. Preferably, the hydrocarbon product(s) include(s) one or more of: alkanes (normal or branched; aliphatic or cyclic), olefins (normal or branched); cyclic aromatics; molecules with combinations of these moieties. Preferably, the oxygenated hydrocarbon product(s) include(s) one or more of: simple alcohols (normal or branched; aliphatic or cyclic), poly-alcohols (normal or branched, aliphatic or cyclic), normal or branched ethers (aliphatic or cyclic), normal or branched poly-ethers (aliphatic or cyclic), simple or poly-ketones (aliphatic or cyclic), simple or poly-aldehydes (aliphatic or cyclic), simple or poly-esters (aliphatic or cyclic), molecules with combinations of these moieties.

Throughout this description, the expression “higher alcohols” denotes an alcohol having two or more carbon atoms (C₂₊ primary, secondary, or tertiary saturated alcohols, or combinations thereof). Similarly, throughout this description, the expression “higher aliphatic hydrocarbon” denotes C₄₊ saturated straight-chain or branched aliphatic hydrocarbons, or combinations thereof.

Throughout this description, the expression “higher value liquid fuel or chemical” denotes a liquid fuel or chemical that is worth more to consumers than the entity to which it is compared. For example, if the process or system starts with a bioprocess intermediate in the form of diluted bioethanol, that diluted bioethanol can be converted to a higher value liquid fuel or chemical by conversion to a liquid fuel, such as a higher alcohol that is worth more than diluted bioethanol. “Worth” in the context provided here denotes overall worth and not simply monetary value (e.g., it takes into consideration efficiency, consumption, environmental value, etc.).

The preferred method and/or system described herein employs a reactive separation unit 1 operation to upgrade a bioprocess intermediate stream 3, or product, to a more valuable liquid fuel or industrial chemical. The method also preferably includes an additional input stream 5 (preferably derived from other carbonaceous or hydrocarbon-containing materials) as a co-reactant to increase the molecular weight and energy density of the product(s) relative to those properties of the starting bioprocess intermediate. The method therefore is capable of capturing chemical or energy value from other sources. The supplemental source(s) may include carbon monoxide, hydrogen, syngas, alcohol(s), or other oxygenated hydrocarbon(s), or any combination of these. The supplemental source may be derived from non-fermentable biomass or other locally available, low-cost materials.

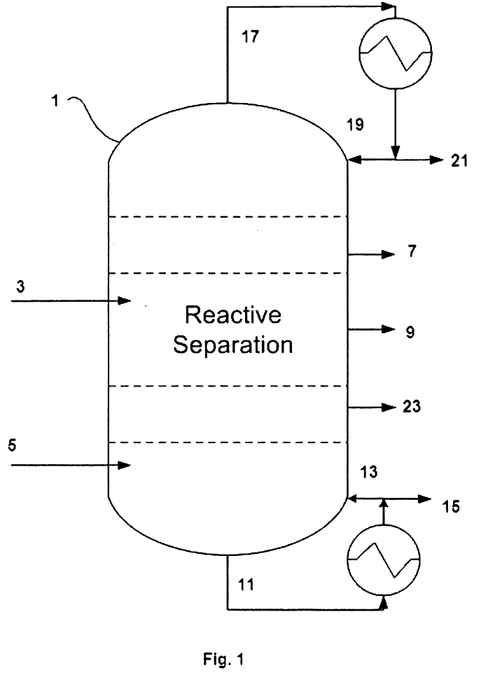

FIG. 1 depicts the various streams useful in the process and system of the invention. A stream 3 containing hydrocarbons or oxygenated hydrocarbons, or an aqueous mixture or solution thereof, can be introduced to the reactive separation unit 1 operation. In the preferred embodiment, the stream 3 is an aqueous solution that includes one or more alcohols or poly-alcohols, and preferably is an intermediate product of fermentation or other bioprocessing operation(s), such as, for example, aerobic and/or anaerobic digestion of organic material, and the like.

An additional reagent or fuel intermediate stream 5 also may be fed to the reactive separation unit 1 in the form of carbon monoxide, hydrogen, syngas, or alcohol(s), other oxygenated hydrocarbon(s), or a combination of any two or more of these. In one preferred embodiment, this additional reagent or feed intermediate stream 5 is a syngas stream of desired and controlled CO and H₂ content. Persons having ordinary skill in the art are capable of determining and controlling the CO and H₂ content of a suitable syngas stream, using the guidelines provided herein.

The two streams may be combined in the reactive separation unit 1 to produce either higher alcohol(s) (C₂+ primary, secondary, or tertiary saturated alcohols, or any combination of these) or higher aliphatic hydrocarbon(s) (C₄+ saturated straight-chain or branched aliphatic hydrocarbons, or a combination of these) product stream(s), or a combination of both products. Alternatively, the two streams may be combined prior to admission to the reactive separation unit 1. In a preferred embodiment, this reactive separation is accomplished by reactive distillation. Using the guidelines provided herein, a person having ordinary skill in the art is capable of carrying out a reactive distillation unit operation on the combined bioprocess intermediate stream 3 and additional input stream 5 to produce a higher value liquid fuel or chemical.

In a further preferred embodiment, the reactive separation unit 1 operation includes a region or stage for slurry-phase, multiphase, or other well-mixed heterogeneous catalytic liquids upgrading reaction(s), which is operated in tandem with the remaining regions or stages of the reactive separations operation.

In another preferred embodiment, water is generated by a variety of possible reaction mechanisms with water rejection, in addition to water that was initially present in the bioprocess stream(s) as a diluent. Preferably, the water is generated within the well-mixed heterogeneous catalytic reaction region or stage of the reactive separation unit 1 operation. In this zone, the desired product molecular weight growth and oxygen removal (as a component of water) are both initiated. The hydrocarbons or oxygenated hydrocarbons are simultaneously concentrated in an organic product phase via this removal of water. Preferably, this well-mixed heterogeneous catalytic reaction region or stage is near the bottom of the reactive separation unit 1 operation when that unit operation is disposed vertically, as shown in the drawings (although vertical orientation is not required). Using the guidelines provided herein, a person having ordinary skill in the art is capable of determining where this well-mixed heterogeneous catalytic reaction region or stage is located depending on the vapor-liquid equilibrium (VLE) behavior of the reacting components, the chemical makeup of the intermediates, temperature, pressure, the composition of the intended product stream(s), as well as engineering associated with tray or stage design and placement and number of stages or trays.

A water-rich stream 23 preferably is disengaged from the organic product phase and purged from the system either immediately at the material inlet stage or region of the reactive separations unit 1 operation, or in a distinct stage or region in a specific location within the reactive separations unit 1 operation. In a preferred embodiment, the exact location of this water-rich draw (i.e., withdrawal of the water-rich stream 23) will depend upon, for example, the vapor-liquid equilibrium (VLE) behavior of the reacting components, reaction intermediates, and the composition of the intended product stream(s), as well as engineering associated with tray or stage design and placement, and the specification of temperature and pressure over the full trajectory of all the stages or regions. The phase separation stage or region thus facilitates removal of a water-rich phase or stream 23 from the reactive slurry or liquid, and the transfer or return of the organic-rich phase to further regions or stages of the reactive separation for continued desired reaction(s) and/or rectification.

In another preferred embodiment, the reactive separation unit 1 operation further incorporates an interstage pressure drop, nozzle arrangement, or isenthalpic flash that facilitates aqueous-organic phase disengagement and separation, and the removal of water or a water-rich phase. This can be situated either at the same location as the well-mixed region or stage, or at an intermediate region or stage in the reactive separation unit 1 operation, i.e., in tandem with organic phase rectification. Interstage pressure drops, specific nozzle arrangements useful in accomplishing the desired disengagement and separation, and isenthalpic flash processes are known to those skilled in the art, who by using the guidelines provided herein, are capable of using such processes or apparatus to produce the desired result. For example, isenthalpic flash processes typically are used in liquefaction of natural gas, as disclosed in, for example, U.S. Pat. Nos. 7,210,311, 7,204,100, 7,010,937, 6,945,075, 6,889,523, 6,742,358, 6,526,777, and 5,615,561, the disclosures of which are incorporated by reference herein in their entirety.

The resultant hydrocarbon-rich phase continues to react in the rectification zone(s) of the integrated reactive separation unit 1 operation, either through the same reactions or additional chain-growth, and/or dehydration reactions. In the preferred embodiment, the reactive separation is accomplished as a reactive distillation—with simultaneous molecular weight increase, oxygen reduction (as a component of water), water removal, and organic product rectification.

In a preferred embodiment, gases are transported upward, by momentum and/or buoyancy, within the reactive separations unit 1 as shown vertically oriented. Overhead vapors 17 are condensed and split as needed into reflux 19 or light product removal and/or purge 21. Likewise at the bottom of the reactive separator, the condensed mixture 11 is sent to the reboiler for return to the column 13 or liquid removal and/or purge 15.

The reactive separation operation(s) allow for two or more boiling point fractions of each product type 7, 9 to be drawn via side streams from the rectification stage(s). The process thus yields higher alcohol(s), liquid hydrocarbon(s), or a combination (and preferably blend) of these chemicals, with a particular application as fuel components. Adjusting product composition through co-feed control strategies, and via controlled combination of the component product cuts, delivers a stand-alone fuel product that can serve as either a replacement or additive to gasoline.

A particularly preferred process upgrades via chemical conversion a bioprocess output stream to higher-value liquids, the higher-value liquids that have utility as liquid fuels, fuel additives, and/or chemical feedstocks, the higher value liquids defined as streams containing organic, aqueous, or mixed-phase (organic/aqueous) aliphatic hydrocarbons (C₄ and above) and/or oxygenated hydrocarbons (C₂ and above), one or more mixture(s) of these components, or a combination of any two or more of these. The preferred process and system provides for conversion of at least a portion of the bioprocess output stream to liquid fuels with simultaneous separation (also known as reaction/separation; also known as reactive separation) of selected size or boiling point product fractions. The preferred process preferably incorporates a second reagent stream, the second reagent stream including carbon monoxide, synthesis gas (“syngas”, primarily a mixture of H₂ and CO), one or more oxygenated hydrocarbon(s), or a combination of any two or more of these reagents, or an aqueous solution or mixture thereof. The relative molar concentrations, or partial pressures, of H₂ and CO in the syngas (H₂ to CO ratio) preferably is controlled to be at a design value selected from within the range of from about 1.0-3.0; more preferably from about 1.5-2.5, and most preferably from about 1.8-2.2. This ratio can be controlled via adjustments upstream of the reaction separation process, specifically by varying the type and adjustable amounts, or relative amounts, of feeds and co-feeds to the upstream syngas generation process.

The combined reaction/separation or reactive separation operation preferably is accomplished via reactive distillation. Reactive distillation methods, systems, and apparatus are well known, and described, for example, in U.S. Pat. Nos. 5,013,407, 5,026,459, 5,368,691, 5,449,801, the disclosures of each of which are incorporated by reference herein in their entirety. Those skilled in the art are capable of designing a suitable reactive distillation method and system for use in providing the combined reaction/separation operation, using the guidelines provided herein.

The preferred process yields one or more of the following product(s) via the indicated mechanism(s): (i) oxygenated hydrocarbons (C₂ and above), achieved via catalytic alcohol condensation with dehydration; (ii) oxygenated hydrocarbons (C₂ and above), achieved via a catalytic aldol coupling reaction mechanism; (iii) aliphatic hydrocarbons (C₄ and above), achieved via a catalytic Fischer-Tropsch reaction mechanism; and (iv) any mix or blend of two or more of these products.

The particularly preferred method and system includes a region within the reactive separation unit for slurry-phase, multiphase, or other well-mixed heterogeneous catalytic liquids upgrading reaction(s), which is operated in tandem with the remaining stages of the reactive separations operations. It is preferred that this embodiment also include a phase separation stage within the reactive separation, in tandem with the slurry-phase or heterogeneous catalytic reaction, which facilitates removal of a water-rich phase from the reactive slurry and return of the organic-rich phase for continued reaction and separations.

Another particularly preferred method and system incorporates an interstage pressure drop, nozzle arrangement, or isenthalpic flash that facilitates aqueous-organic phase separation and removal of water or a water-rich phase from the reactive separation operation. Other preferred processes and systems include incorporating interstage pressure drops, and an overall pressure profile over the path of the reactive separations stages, which facilitates removal of water or a water-rich phase from an intermediate stage in the reactive separation operation, i.e., in tandem with organic phase rectification. Other preferred processes incorporating interstage pressure drops, water takeoff(s), and overall pressure and temperature profiles over the path of the reactive separations stages that yield the intended product stream(s) at the design product take-off location(s), on the basis of the tendency toward vapor-liquid equilibrium at each of the stages within the reactive separations operation.

Particularly preferred and exemplary embodiments now will be described with reference to the following non-limiting examples.

### EXAMPLE 1

### Production of isobutanol

Isobutanol (also 2-methyl-1-propanol; i-C₄H₉OH, hereinafter i-BuOH), can be produced from an aqueous unrefined ethanol intermediate stream 3, and a syngas 5. A 41% aqueous ethanol (“EtOH”), as is typically generated from corn-based carbohydrate fermentation via alcohol generation and primary separation of some water and dried distiller's grains and solubles (“DDGS”) in a separations unit, is available as a feedstock at a nominal quantity of about 50 Mgpy (50,000,000 gallons per year), on an EtOH-only basis. This liquid solution is introduced as-is to the reactive separations operation 1. Synthesis gas, or syngas stream, is generated separately, and also introduced to the reactive separation operation 1, at a H₂/CO ratio of 2.0, and two molar equivalents relative to the feed EtOH. Thus the starting materials have the relative mole ratio: 1 EtOH/ 2 CO/ 4 H₂.

On these bases, the combined feed to the reactive separation unit is approximately as follows:

17,046 kg/hr EtOH with 24,529 kg/hr water - at 70 C. and 1 atm,
pumpable to the pressure of the reactive separations operation (60 atm);
2,987 kg/hr H₂ - at 400 C. and 60 atm;
20,728 kg/hr CO - at 400 C. and 60 atm.

The reactive separations unit is operated at 300 C and 60 atm. The overall reaction in this case is:

2CO+4H₂+C₂H₅OH=i−C₄H₉OH+2H₂O

Thermodynamically, this reaction is slightly reversible, but largely favored over the full range of temperatures of interest—and also enhanced (shifted, to the right) with higher pressure. Specifically at the conditions cited, the equilibrium constant for this overall reaction at 300 C is calculated as 1.43×10³, using the commercially-available package HSC Chemistry® 6.0, and specifically referencing the pure component formation energies and enthalpies as provided by its well-established databases. See Roine, A., HSC Chemistry® 6.0, Outokumpu Technology, Pori, Finland; ISBN-13: 978-952-9507-12-2; August 2006.

Because the reaction results in a decrease in the number of gas-phase moles (by 4, as written) this equilibrium constant is in units of [bar⁻⁴], which reflects also the potential impact of pressure on product distribution. This influence is intermediate in the present case, relative to the extremes of syngas only for i-BuOH synthesis (mole difference=8), and alcohol homologation without syngas—or “Guerbet synthesis” (mole difference=0).

As is standard for equilibrium constant calculations and application, this does not take into account transport or kinetic effects, or the influence (via relative kinetics) of competing reactions. For simplicity of illustration, this single product (i-BuOH) is assumed. The reaction stoichiometry applied here reflects an equal contribution of carbon number from the two sources—fermentation and syngas intermediates.

The combined influence of the equilibrium constant and the pressure effect gives rise to a one-pass (equilibrium) conversion—or limiting one-stage extent of reaction—of 0.97 for this net reaction. The overall yield can be improved to, and even beyond this limit, because of the continuous separation of products, and reflux of reactants—as well as the multistage action with equilibrium approached at each stage. More conservatively here, allowing for losses and/or byproducts, a total conversion of 0.95 is assumed for the targeted reaction.

With these assumptions and the attendant conversion and mass balance calculations, a product stream of 26,055 kg/hr i-BuOH with 43,860 kg/hr water, corresponding to 37.3% i-BuOH, is taken as a column side draw. This is amenable to recovery by simple azeotropic distillation, by close analogy to similar systems. See Luyben, W. L., “Control of the Heterogeneous Azeotropic n-Butanol/Water Distillation System”, Energy & Fuels, 22 (6), 4249-4258, September 2008.

By means of this process, the energy generated by the reactive separations exotherm is enough to fully drive that process, with the complete vaporization of the product stream (at 300 C and 60 atm), and also provide some excess energy for other use. Assuming vapor phase products (both i-BuOH and water) at the system temperature of 300 C, this excess energy available is approximately 7900 Mcal/hr (=31.3 MMBTU/hr=9.2 MWₜₕ). This can be applied toward the residual azeotropic separations burden which should be small, or even negative in this case (starting with the relatively hot vapor stream), or a primary fermentations separation operation (upstream, if applicable), or other preheating functions (limited by the 300 C energy quality).

This isobutanol product has wide utility as a chemical intermediate in the synthesis of coatings, and flavor and fragrance agents. Its primary derivative is isobutyl acetate for these applications. Isobutanol also has direct utility as a solvent, plasticizer, and chemical extractant. Additionally, it has utility as a fuel additive and de-icing agent.

### EXAMPLE 2

### Production of 1-hexanol

The production of 1-hexanol ((also hexyl alcohol; n-hexanol; n-C₆H₁₃OH; here “H×OH”), is accomplished from an aqueous (unrefined) ethanol intermediate 3, and syngas stream 5, using the second mode of operation of unit 1 as described above, which includes a pressurized feed/lowest stage(s); pressure letdown (e.g., flash) to upper, lower pressure, vapor only stages. The same 41% aqueous ethanol (“EtOH”) solution, and syngas, in the same relative molar equivalents and mole ratios as used in Example 1 above is used in this example. On these bases, the combined feed to the reactive separation unit is approximately as follows:

17,046 kg/hr EtOH with 24,529 kg/hr water - at 70 C. and 1 atm,
pumpable to the pressure of the lower section (see below) of the
reactive separations operation (here, 80 atm);
2,987 kg/hr H₂ - at 400 C. and 80 atm;
20,728 kg/hr CO - at 400 C. and 80 atm.

The reactive separations unit 1 is operated under position-dependent conditions, consistent with the operating concept of the second mode of operation described above. The lower section is maintained at saturated or sub-saturated conditions with respect to aqueous vapor pressure, and is thus a multi-phase slurry: aqueous reactants, products, and solid catalyst. Here, these bottom 2 stages (i.e., lower section) are maintained at 280 C and 80 atm.

An intermediate, water-rich phase is removed from the bottom section (stage 2), phase-separated, and the water-rich component is re-injected to the bottom section (stage 1). An intermediate organic-rich phase is reduced in pressure (flashed) and directed to the remaining stages of the reactive separation. The remaining stages (upper section) are operated at a lower pressure, and higher temperature—the latter chosen to (a) maintain vapor-phase operations in this section; (b) enhance reaction kinetics; (c) to capture the contributions of straight-chain (as opposed to branched) higher alcohol synthesis reaction mechanisms. The latter effect has been described by Olson et al., and gives rise to the potential for H×OH production in this operating mode. Olson, E. S., R. K. Sharma and T. R. Aulich, “Higher Alcohols Biorefinery—Improvement of Catalyst for Ethanol Conversion”, Applied Biochemistry and Biotechnology, 115; 913-932 (2004).

Here, the upper section is operated at 350 C and 20 atm. The overall reaction in this case is:

3CO+6H₂+1.5C₂H₅OH=n−C₆H₁₃OH+3.5 H₂O

Thermodynamically, this reaction is only slightly reversible; it is largely favored over the full range of temperatures of interest—and also enhanced (shifted, to the right) with higher pressure. Specifically at the conditions cited, the equilibrium constant for this overall reaction at 280 C and 350 C is calculated as 1.50×10⁸ and 8.29×10², respectively, using the commercially-available package HSC Chemistry® 6.0, and specifically referencing the pure component formation energies and enthalpies as provided by its well-established databases. See Roine, A., HSC Chemistry® 6.0, Outokumpu Technology, Pori, Finland; ISBN-13: 978-952-9507-12-2; August 2006.

Because the reaction results in a decrease in the number of gas-phase moles (by 6, as written) this equilibrium constant is in units of [bar⁻⁶], which reflects also the potential impact of pressure on product distribution. This influence is intermediate in the present case, relative to the extremes of syngas only for H×OH synthesis (mole difference=12), and alcohol homologation without syngas—or “Guerbet synthesis” (mole difference=0).

As is standard for equilibrium constant calculations and application, this does not take into account transport or kinetic effects, or the influence (via relative kinetics) of competing reactions. For simplicity of illustration, this single product (H×OH) is assumed. The reaction stoichiometry applied here reflects an equal contribution of carbon number from the two sources—fermentation and syngas intermediates.

The combined influence of the equilibrium constant and the pressure effect gives rise to a one-pass (equilibrium) conversion—or limiting one-stage extent of reaction—of 0.96 for this net reaction. The overall yield can be improved to, and even beyond this limit, because of the continuous separation of products, and multistage operations with equilibrium approached at each stage. More conservatively here, allowing for losses and/or byproducts, a total conversion of 0.95 is assumed for the targeted reaction.

With these assumptions and the attendant conversion and mass balance calculations, a product stream of 23,944 kg/hr H×OH with 45,971 kg/hr water, corresponding to 34.2% H×OH, is taken as a column side draw. This is amenable to recovery by simple azeotropic distillation, by close analogy to similar systems. See Luyben, W. L., “Control of the Heterogeneous Azeotropic n-Butanol/Water Distillation System”, Energy & Fuels, 22 (6), 4249-4258, September 2008.

By means of this process, the energy generated by the reactive separations unit 1 exotherm is enough to fully drive that process, with the complete vaporization of the product stream (at 350 C and 20 atm), and also provide some excess energy for other use. Assuming vapor phase products (both H×OH and water) at the system temperature (upper section) of 350 C, this excess energy available is approximately 6280 Mcal/hr (=24.9 MMBTU/hr=7.3 MWₜₕ). This can be applied toward the residual azeotropic separations burden which should be small, or even negative in this case (starting with the relatively hot vapor stream), or a primary fermentations separation operation (upstream, if applicable), or other preheating functions (limited by the 350 C energy quality).

This n-hexanol product has wide utility as a chemical intermediate; its primary derivatives are esters, for applications in the synthesis of pharmaceuticals, antiseptics, and flavors and fragrances. Additionally, n-hexanol has potential utility as a fuel or fuel additive.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, this disclosure is intended to be illustrative, but not limiting of the scope of the invention.

What is claimed is:

1. A chemical conversion process that converts a bioprocess output stream to higher-valve liquids, the process comprising:
introducing the bioprocess output stream into a reactive separation unit, the bioprocess output stream comprising at least one component selected from the group consisting of a hydrocarbon product, an oxygenated hydrocarbon product, and mixtures thereof;
introducing a second stream into the reactive separation unit, the second stream comprising at least one component selected from the group consisting of carbon monoxide, hydrogen, syngas, alcohols, oxygenated hydrocarbons, and mixtures thereof;
combining the bioprocess output stream and the second stream; and
subjecting the combined streams to reactive separation to produce at least one product selected from the group consisting of higher alcohols, higher aliphatic hydrocarbons, and mixtures thereof,
thereby converting at least a portion of the bioprocess output stream to higher value liquid fuels or chemicals by reaction and separation of selected size fractions or boiling point product fractions of the bioprocess output stream.

2. The process of claim 1, wherein the first stream is an aqueous solution comprising one or more alcohols or poly-alcohols.

3. The process of claim 2, wherein the aqueous solution is an intermediate product of fermentation or other bioprocessing operations.

4. The process of claim 1, wherein the second stream is a syngas stream comprising CO and H₂.

5. The process of claim 4, wherein the relative molar concentrations of H₂ and CO in the syngas (H₂ to CO ratio) is within the range of from about 1.0 to about 3.0.

6. The process of claim 1, wherein the reactive separation is accomplished by reactive distillation.

7. The process of claim 1, wherein subjecting the combined streams to reactive separation produces an oxygenated hydrocarbon product, the oxygenated hydrocarbon product produced by one or more processes selected from the group consisting of catalytic alcohol condensation with dehydration, and catalytic aldol coupling reaction.

8. The process of claim 1, wherein subjecting the combined streams to reactive separation produces higher aliphatic hydrocarbons, the aliphatic hydrocarbons produced by a catalytic Fischer-Tropsch reaction.

9. The process of claim 1, wherein subjecting the combined streams to reactive separation produces both higher alcohols and higher aliphatic hydrocarbons through parallel reactive separation schemes, in which and a first reactive separation yields primarily liquid aliphatic hydrocarbons, and a second reactive separation yields primarily liquid higher alcohols.

10. The process of claim 9, wherein the primarily liquid aliphatic hydrocarbons from the first reactive separation and the primarily liquid higher alcohols from the second reactive separation are combined in a desired ratio.

11. The process of claim 1, further comprising:
operating slurry-phase, multiphase, or other well-mixed heterogeneous catalytic liquid upgrading reactions in a region in the reactive separation unit in tandem with the remaining portions of the process.

12. The process of claim 11, further comprising:
operating phase separation in a region in the reactive separation unit, the phase separation operated in tandem with the slurry-phase, multiphase, or other well-mixed heterogeneous catalytic liquid upgrading reactions, wherein the phase separation facilitates removal of a water-rich phase from reactive slurry and return of organic-rich phase for continued reaction or rectification.

13. The process of claim 11, further comprising:
separating an aqueous-organic phase mixture and removing water or a water-rich phase from the reactive separation unit through one or more processes selected from the group consisting of an interstage pressure drop, nozzle arrangement, and isenthalpic flash.

14. The process of claim 11, further comprising:
removing water or a water-rich phase from the reactive separation unit during an intermediate stage in which the interstage pressure drops and the overall pressure profile over the path of the reactive separations facilitates the removal.

15. The process of claim 1, wherein the bioprocess output stream comprises at least dilute bioethanol.
